(19) United States
(12) Patent Application Publication
(10) Pub. No.: US 2024/0050658 A1
(43) Pub. Date: Feb. 15, 2024

(54) AUTOINJECTOR WITH GAS

(71) Applicant: Sanofi, PARIS (FR)

(72) Inventors: Ramsay Black, Melbourn, Hertfordshire (GB); Uwe Dasbach, Frankfurt am Main (DE); Thomas Mark Kemp, Melbourn, Hertfordshire (GB); William Timmis, Melbourn, Hertfordshire (GB)

(21) Appl. No.: 18/267,055

(22) PCT Filed: Dec. 16, 2021
(86) PCT No.: PCT/EP2021/086127

§ 371 Date: Jun. 13, 2023

(30) Foreign Application Priority Data
Dec. 17, 2020 (EP) 20315496.8

(51) Int. Cl. A61M 5/20

(52) U.S. Cl. CPC A61M 5/2046

(57) ABSTRACT

The invention relates to an autoinjector (100) comprising —a housing (170) which is gas-tight to the outside, —a fluid reservoir (110) with a fluid, wherein the fluid reservoir (110) comprises a flexible material, —a container (140) which comprises at least a first section which is configured to contain a first substance which comprises gas or which can be converted into gas, wherein ⋅the container (140) is configured to assume two different states, ⋅a first state in which the first section is sealed so that the at least first substance is contained in the first section, ⋅a second state in which the at least first substance is released from the first section into the housing (170) causing an increase of gas pressure within the housing (170) which acts on the fluid reservoir (110) causing the fluid inside the fluid reservoir (110) to move through the fluid reservoir (110) towards and through an outlet (230) of the autoinjector (100).

### FIELD OF INVENTION

The present disclosure relates to an autoinjector.

### BACKGROUND OF INVENTION

In regular drug delivery devices, a single drive mechanism may be housed in a housing of the drug delivery device and is used in conjunction with several cartridges, syringes or ampules to dispense drug contained in the cartridge, syringe or ampule from the device.

Devices of this kind, however, are designed for housing a cartridge or a syringe, and, due to the shape of these components, the overall shape of the device is adapted to the syringe, cartridge or ampule, as they are rigid components. This is usually decisive or at least limiting for the form factor of the device.

### SUMMARY

It is an object of the present disclosure to provide an alternative autoinjector. This object is solved by the present disclosure and, particularly, by the subject-matter of the independent claim. Advantageous embodiments and refinements are subject to the dependent claims.

The disclosure relates to an autoinjector comprising a housing which is gas-tight to the outside, a fluid reservoir with a fluid, wherein the fluid reservoir comprises a flexible material. The housing further comprises a container which comprises at least a first section which is configured to contain a first substance which comprises gas or which can be converted into gas. The container is configured to assume two different states, a first state in which the first section is sealed so that the at least first substance is contained in the first section, a second state in which the at least first substance is released from the first section into the housing causing an increase of gas pressure within the housing which acts on the fluid reservoir causing the fluid inside the fluid reservoir to move through the fluid reservoir towards and through an outlet of the autoinjector. In this way the movement of the fluid towards the outlet is triggered by an increase of a gas pressure inside the housing of the autoinjector. The change from the first state to the second state of the container triggers then the increase of the gas pressure inside the housing.

The first substance may be pressurized gas or a liquefied gas with a high vapour pressure which converts into gas at a certain temperature, for example room temperature.

Using a flexible fluid reservoir has the advantage to have some degree of freedom to choose the shape of the reservoir. For example it can have a different shape than a syringe.

In an embodiment the fluid reservoir is arranged circumferentially around a longitudinal axis, wherein the reservoir is oriented along a circle segment. In an embodiment the reservoir comprises a pouch or another hollow shaped body which is configured for moving fluid through it. The pouch can be collapsible.

Employing a flexible reservoir has the advantage of improved robustness, in particular compared to a glass syringe which is fragile and can break. Further, there is an improved drug integrity and less contamination risk, because the reservoir only comprises one opening, which needs to be sealed, which is on the injection side. In autoinjectors with syringes, for example, there is additionally the side of the stopper to be sealed.

Another advantage is the opportunity for a different form with usability benefits, because the reservoir can be adjusted to the form of the device. Its compactness and consistency avoids large pre-filled syringe (PFS) tolerances reducing injection variability. In particular it is possible to employ plastic instead of glass for the reservoir which can be manufactured with a higher precision compared to glass. It further has a reduced stalling risk and no stopper friction, because there is no stopper required. The reservoir can for example be filled by vacuum filling to eliminate any air, or steam purging prior to container closure. In the case of vacuum filling the pouch can be pulled apart (e.g. by a vacuum) which creates a vacuum inside the pouch. This pulls liquid from a connected container inside the pouch.

In an embodiment the reservoir comprises a narrowing portion which connects the reservoir and the delivery tube. The narrowing portion ensures that the fluid is forced into the delivery tube, and the amount of fluid which is not released from the reservoir is reduced to a minimum.

In an embodiment the container comprises a gas vessel with a shape of a hollow cylinder segment with the longitudinal axis being its main axis, and having a cut-out along the longitudinal axis on its circumference. Such a shape has the advantage that available and accessible space which is arranged radially inwards of the inner wall of the cylinder can be used for arranging further components, so that the overall size of the device can be kept small.

In an embodiment the container comprises a second section which is configured to contain a second substance which can be converted into gas, wherein the first section and the second section are separated by a separation member, such that when the separation member is opened the first substance, which comprises a substance which can be converted into gas, mixes with the second substance and generates gas. The first substance may be liquid or solid. Likewise may the second substance be liquid or solid. It needs to be ensured that the first and second substance can be mixed and that gas is generated when they are mixed. For example the first substance may be citric acid and the second substance sodium bicarbonate.

The liquefied gas may comprise a vapour pressure so that at a certain temperature, for example at room temperature, the fluid boils and at least partially converts into gas.

In an embodiment the container comprises a gas sealing which seals the first and/or second section and which is opened when switching from the first state to the second state. The gas sealing may comprise a plastic material or a welded metal lid. A sealing according to the current disclosure has the advantage that it can be more easily opened compared to a vessel so that gas can emerge and increase the pressure inside the housing.

In an embodiment the separation member is configured to be opened during assembly of the autoinjector or by a tool. Preferably the separation member is opened before the gas sealing is opened, so that gas has been already generated when the gas sealing is opened and the gas is released into the housing.

In an embodiment the autoinjector comprises a trigger which is movable along a longitudinal axis from a first trigger position to a second trigger position, wherein
in the first trigger position the trigger is separated from the gas sealing,
in the second trigger position the trigger is in mechanical contact with the gas sealing, thereby opening the gas sealing, such that gas is releasable from the container into the housing and thereby increasing the gas pressure inside the housing, such that the fluid reservoir is squeezed and the fluid inside the fluid reservoir is moved to the outlet. The trigger can be pushed by a user, in particular a patient, from the first position to the second position. In this way the user can determine when the gas pressure inside the housing increases and subsequently when the fluid is moved to the outlet.

In an embodiment the autoinjector comprises a trigger sealing which is arranged between the housing and the trigger, such that the housing is gas-sealed. The sealing ensures also that the housing remains gas-sealed while the trigger moves from the first to the second position.

In an embodiment the autoinjector comprises a trigger spring which is mechanically connected to the trigger such that when the trigger moves from the first position to the second position the trigger moves the trigger spring from a first position to a second position.

The trigger spring may be is expandable and compressible along the longitudinal axis, such that when the trigger moves from the first position to the second position the trigger moves the trigger spring from an expanded status to a compressed status, thereby acting against the force of the trigger spring.

The trigger spring ensures that the trigger is held in its starting position which aligns the trigger the outer surface of the housing and providing a continuous outer housing surface, unless the trigger is pressed. It is then some force against the trigger spring required in order to move the trigger from the first position into the second position.

The trigger spring may comprise a pressure, a compression spring, a torsion spring or a tension spring. The trigger spring which may comprise metal.

In an embodiment the autoinjector comprises a delivery tube which is configured to be in fluid communication with the fluid reservoir and which is configured to be in fluid communication with the outlet, such that fluid from the fluid reservoir is movable from the fluid reservoir through the delivery tube to the outlet. This ensures more flexibility as the delivery tube can guide the flow of fluid in a predetermined direction. The delivery tube may comprise a flexible material, such as an elastomer or a stiff material, such as a plastomer or thin metal.

In an embodiment the autoinjector comprises an outlet interface which is movable relative the longitudinal axis from a first interface position to a second interface position, and which is in fluid communication with the outlet. In the first interface position the outlet interface is not in fluid communication with the delivery tube, whereas in the second interface position the outlet interface is in fluid communication with the delivery tube. By moving the outlet interface it can be controlled if a fluid communication between the fluid reservoir and the outlet is established. This fluid communication is only established if the trigger is pushed, so that the seal opens and the gas increases the pressure inside the housing.

In an embodiment the autoinjector comprises an interface spring which is mechanically connected to the outlet interface.

The interface spring may be expandable and compressible along the longitudinal axis, such that it is more compressed in the second tube position as in the first tube position. This ensures that the outlet interface is not in fluid communication unless a pressure causes it to move against the force of the interface spring towards the base of the housing to a position where it is in fluid communication with the fluid reservoir.

In an embodiment the outlet comprises a needle, which is movable along the longitudinal axis from a first needle position to a second needle position, wherein in the first needle position the needle is completely contained in the housing, and in the second needle position at least a portion of the needle has moved through a housing sealing which is arranged at the housing around the longitudinal axis.

In an embodiment the trigger is integrated into the housing, such that a part of the outer surface of the housing comprises the trigger. In this way the function of initiating the injection process is integrated into the housing.

In an embodiment the housing may have a shape with a base which has a larger diameter than the height, which extends along the longitudinal axis. In an embodiment the shape comprises a cylinder, in particular a cylinder with rounded edges.

In an embodiment the fluid reservoir comprises a medicament or drug.

In an embodiment the autoinjector is a disposable or single-use device, for providing a single dose.

In an embodiment, the fluid reservoir is arranged at a base of the housing along a circle segment. The fluid reservoir may have essentially the shape of a torus segment. For example, the fluid reservoir extends by at least 180° or at least 200° or at least 270° along the circle segment. For example, the fluid reservoir is arranged, at least partially, circumferentially around the longitudinal axis. The longitudinal axis may run through the centre of the circle of the circle segment.

In an embodiment, the container is arranged inside the housing. The container may be arranged such that it is, at least partially, circumferentially surrounded by the fluid reservoir. For example, the longitudinal axis runs through the container or the container is arranged radially between the longitudinal axis and the fluid reservoir.

The container may have the shape of a hollow lateral side of a cylinder. Thus, the interior of the container may be limited in radial direction by two opposing walls of the container having shapes of a cylinder lateral surfaces with different radii.

The container may circumferentially surround the trigger spring and/or the outlet interface and/or the needle. The container may be radially arranged between the fluid reservoir and the trigger spring or the outlet interface or the needle, respectively.

### BRIEF DESCRIPTION OF THE DRAWINGS

Embodiments will now be described, by way of example only, with reference to the accompanying drawings, in which:

FIG. 1 shows a cross-section from an angled top view of an autoinjector.

FIG. 2 shows a side view of a cross-section from an autoinjector.

FIG. 3 shows a housing sealing.

FIG. 4A shows an angled top-view of the of a button.

FIG. 4B shows the button in a 3D side view.

FIG. 5 shows a diagram temperature on vapour pressure for common refrigerants

### DETAILED DESCRIPTION

The same reference numbers apply to the same features throughout the figures and the following explanations.

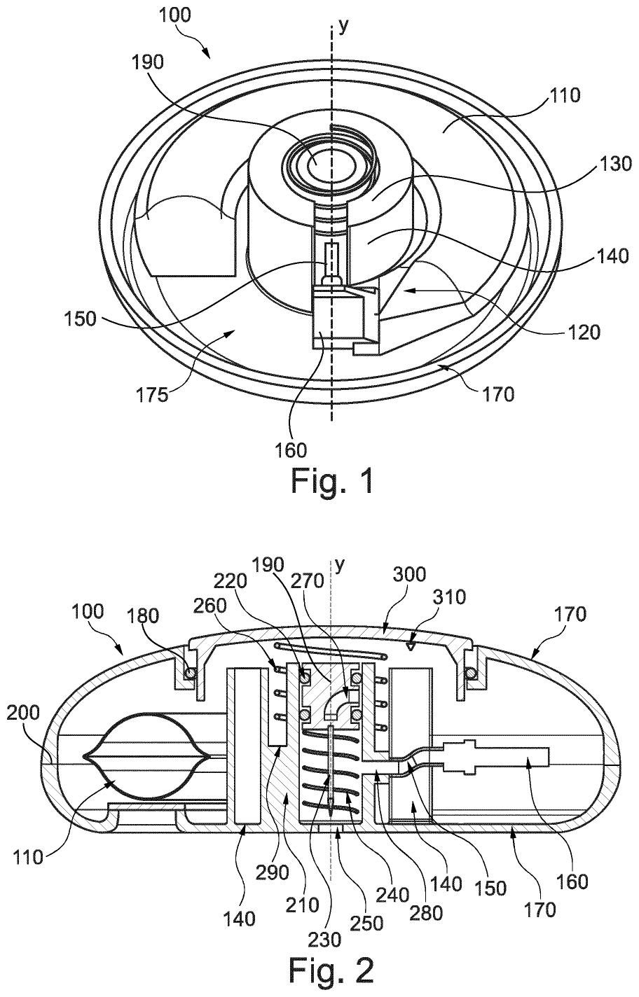

FIG. 1 shows a cross-section from an angled top view of an autoinjector. It is shown a housing 170 with a circular cross-sectional shape and a base 175. Other shapes are possible, of course.

Inside of the housing 170 a pouch 110 is arranged at the base 175 around a longitudinal axis y arranged at a cross-section of a circle element. The pouch 110 contains a fluid material, e.g. a liquid medicament or medicament formulation. One ending of the pouch 110 is in fluid communication with a needle 230. This ending comprises a narrowing portion 120, which is in fluid communication with a delivery tube 150. The delivery tube 150 is configured to be in fluid communication with the needle 230. There can be provided an additional spout 160 which connects the delivery tube 150 and the narrowing portion 120. The spout 160 enables a coupling between the pouch 110 and the delivery tube 150. The spout 160 has different material properties than the pouch 110. For example it is more stiff so that the alignment of the pouch 110 in the housing 170 is based on the position and orientation of the spout 160.

A gas vessel 140 is arranged around the longitudinal axis y. The gas vessel 140 is arranged inside the housing, radially between the pouch 110 and the longitudinal axis y and has a shape of a segment of a hollow cylinder, such that the cross-section comprises a circle segment. The hollow cylinder section comprises an inner and an outer wall which form a cavity in which the pressurized gas is contained. The opening also extends axially along the longitudinal axis y. The gas vessel 140 comprises a gas sealing 130 which is arranged at one ending of the hollow cylinder segment covering its cross-sectional area. This ending might be opposite to the ending at the base 175 with respect to the longitudinal axis y. The gas sealing 130 can comprise a plastic material or a welded metal lid. The gas sealing 130 may cover the entire cross-section of the circle segment. Along the inner wall of the hollow cylinder segment a button spring 260 is arranged around the longitudinal axis y, which is expandable and compressible along the longitudinal axis y and which is mechanically connected to a button 300 (not shown here, see FIG. 2, for example).

The gas vessel 140 can comprise a polymer vessel, for example a flouropolymer, which can contain gas with a pressure of 40 to 60 bar, in particular 50 bar. The vessel can comprise a very small size of the order of 0.5-1.5 ml, in particular 1 ml.

Further a needle carrier 190 is arranged around the longitudinal axis y. The needle carrier 190 is arranged radially inward in relation to the gas vessel 140. A needle 230 is attached to the needle carrier 190. The delivery tube 150 is arranged angled to the narrowing portion 120 and directed to the longitudinal axis y, and thereby passing through the open part in circle segment of the gas vessel 140, from the outer surface of the gas vessel 140 which faces the inner surface of the housing 170 to the region delimited by an inner surface of the gas vessel 140. The inner surface and the outer surface may face into opposite directions. The gas may be retained in the space defined between the inner surface and the outer surface.

FIG. 2 shows a side view of a cross-section of the autoinjector 100. A holding portion 210 is arranged around the longitudinal axis y, and comprises a cylindrical portion. The holding portion 210 is fixed to the base 175 of the housing 170. Inside the cylindrical portion of the holding portion 210 the needle carrier 190 is arranged. At the housing 170 a button 300 is arranged which is movable along the longitudinal axis y towards the gas sealing 130 of the gas vessel 140, the holding portion 210 and the needle carrier 190. The holding portion 210 and the gas vessel 140 are mechanically connected by a bridge 290 which extends around the longitudinal axis y. A button spring 260 is radially arranged between the housing portion 210 and the inner wall of the hollow cylinder segment of the gas vessel 140. The button spring 260 is expandable and compressible along the longitudinal axis y between the bridge 290 and the button 230. The trigger spring may comprise a pressure, a compression spring, a torsion spring or a tension spring. The trigger spring which may comprise metal.

The holding portion 210 further comprises a tube interface 280 which is in fluid connection with the delivery tube 150. The tube interface 280 is arranged axially between the bridge 290 and the base 175 On the radial outward side of the needle carrier 190 needle carrier sealings 220 are arranged, which are in contact with a radial inner side of the holding portion 210.

The needle carrier 190 comprises a needle tube 270 which is fluidly connectable to the delivery tube 150. A needle 230 is mechanically fixed to the needle carrier 190 and is in fluid communication with the needle tube 270.

The needle tube 270 is also an fluid communication with a needle 230, which is arranged along the longitudinal axis y.

A tube spring 240 is arranged axially between the needle carrier 190 and the base of the housing 170 with respect to the longitudinal axis y inside the cylindrical portion of the holding portion 210, and which is expandable and compressible along the longitudinal axis y.

The button 300 comprises at least one piercing spike 310 which is directed to the inside of the housing 170 and towards the gas sealing 130. Instead or additionally to one or more piercing spikes 310 the button 300 may comprise other sharp elements which are configured to open the gas sealing 130. The housing 170 may comprise two parts which are joined together. In this case the housing can comprise an additional sealing part 200 where the two parts are joined together, for example by gluing or soldering. The sealing part 200 may comprise a region with increased thickness which increases the overall stability at the region where the two parts are joined together. The button 300 is movable along the longitudinal axis y to the inside of the housing 170 against the force of a button spring 260. A button sealing 180 is axially arranged between the housing 170 and the button 300, and ensures that the housing 170 remains gas-tight towards the outside, also when the button 300 moves along the longitudinal axis y. The housing 170 needs to be designed such that it remains gas-tight even if there is a failure of the gas sealing 130. At the base 175 of the house 170 a housing sealing 250 is arranged around the longitudinal axis y.

In the following the function of the autoinjector 100 is described.

A user may press the button 300 axially in the direction towards the housing 170 along the longitudinal axis y. At some point during the movement the piercing spikes 310 damage the gas sealing 130 so that pressurized gas can emerge from the gas vessel 140. The gas distributes within the housing 170 which is gas-tight towards the outside. The overall gas pressure within the housing 170 increases. This leads to a squeezing of the pouch 110, so that the fluid inside the pouch 110 is moved through the narrowing portion 120, through the delivery tube 150 and to the tube interface 280.

Due to the increased gas pressure inside the housing 170 the needle carrier 190 is pushed along the longitudinal axis y towards the base 175 of the housing 170. Along with the needle carrier 190 the needle tube 270 and the needle 230 move towards the base 175. When the needle carrier 190 cannot move further in the direction towards the base 175 the needle tube 270 and the tube interface 280 are aligned so that they are in fluid communication. The tube interface 280 comprises the same cross-section as the needle tube 270. The fluid, which moves through the delivery tube 150 is then moved through the tube interface 280, through the needle tube 270 to the needle 230. The needle 230 which has been also moved downwards along with the needle carrier 190 has further progressed partially through a housing sealing 250 to the outside of the housing 170 for an injection.

Since the internal volume of the housing 170 is larger than the volume of the pouch 110, the driving pressure towards the fluid is relatively constant over the course of the injection.

The gas may comprise CO₂ at low to moderate pressures (at pressures >57 bar it condenses into a liquid and acts as a saturated mixture), N₂, air or Nitrous oxide. The gas should be non-toxic, non-flammable and available at low costs.

The pouch 110 may consists of more than one material which may be specific adaptable to its inside and outside according to chemical and/or mechanical requirements. For example regarding the outside of the pouch 110 a material is needed which does not engage in a chemical reaction with the gas in the gas vessel 140, and which is mechanically stable with regard to the pressure applied to the pouch 110 due to the emerging gas of the gas vessel 140. Regarding the inside of the pouch 110 a material is required which does not engage in a chemical reaction with the fluid contained in the pouch 110.

In another embodiment (not shown here) instead of a gas vessel a container 140 is used which comprises two sections, each of which contains a liquid or solid substance which generate gas when they are mixed. The sections are separated by a separation member. When the separation member is opened the substances mix and thereby generate gas. When the gas is released from the container 140 to the inside of the housing 170 it acts on the pouch 110. The pressurized gas causes the squeezing of the pouch 110, as described above. Exemplary substances which may be mixed to generate gas are sodium bicarbonate and citric acid: Preferably non-toxic reactants are used which produce safe products, with CO₂ being the gas produced. Selection criteria for the fluids may be chemical compatibility of the pouch with reactants and products, temperature dependence of the reactants and products, for achieving a constant reaction rate (affected by particle size), non-toxic and not significantly exothermic or endothermic.

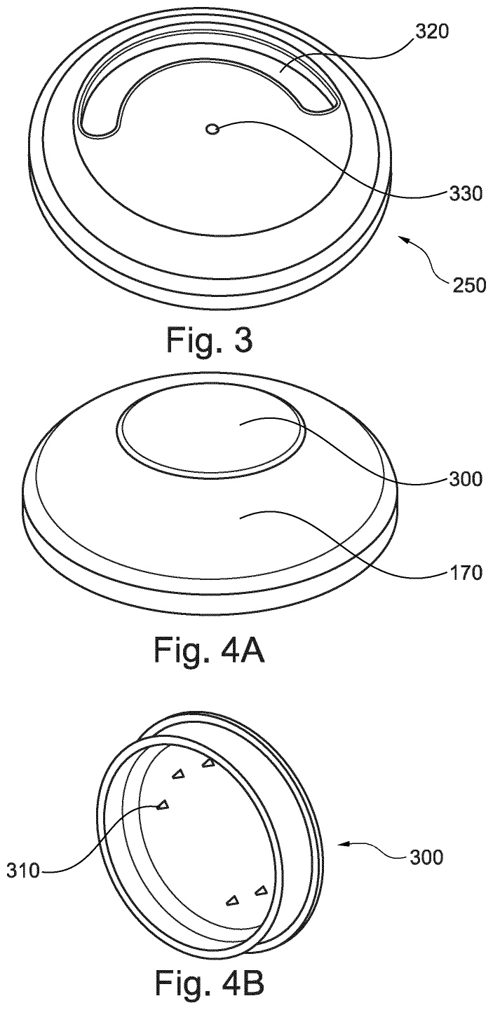

FIG. 3 shows a housing sealing 250 with a circular shape. In the centre a needle hole 330 is indicated, where the needle 230 can progress through during an injection operation. Further a window 320 is shown through which for example the container can be viewed visually prior to injection with regard to its integrity, for visually checking the drug for blurred or diluted regions, for tracking the injection process, and for visually confirming that the entire dose has been delivered.

FIG. 4A shows an angled top-view of the of the button 300 and the part of the housing 170 where it is integrated. The button 300 has a circular shape, but it may has also another shape such as rectangular or quadratic.

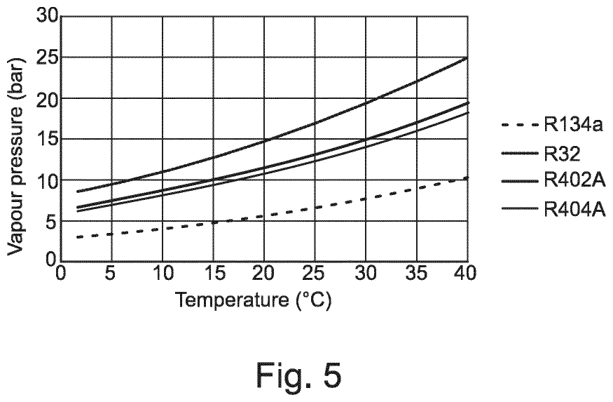

FIG. 4B shows the button 300 in a 3D side view. The button 300 comprises a cylindrically formed frame and at its inside piercing spikes are arranged In another embodiment (not shown here) instead of a gas vessel a container 140 is used which is configured to contain a liquid with a high vapour pressure such that gas is generated inside the container 140 when a certain temperature has been reached. FIG. 5 shows a diagram temperature on vapour pressure for common refrigerants, such as R134a, R32, R402A or R404A. which can be used in this embodiment. Those refrigerants can be contained in the container 140, turning into gas at a certain temperature. They should have a vapour pressure which is near to the required driving pressure. The container 140 comprises a gas sealing 130.

The gas can then be released from the container 140 into the housing 170 by opening the gas sealing 130 as described in FIG. 2. In this embodiment possible gases are refrigerants, such as R134a, CO₂ at high pressures, or alkanes such as propane or butane may be used. Selection criteria for gases may be that the vapour pressure may be at a temperature where the device is used such as room temperature, sensitivity of vapour pressure to temperature, diffusion (related to molecular size).

The device may have a height between 10-40 mm, and in particular a height between 15-30 mm. The base of the device may have a diameter between 45-90 mm, and in particular a diameter between 50-70 mm. In particular the height of the device may be smaller by a factor of more than three compared to a typical autoinjector comprising a syringe. This is advantageous for a user like a patient, because the distance from the skin to the position where the device is triggered is much less.

The scope of protection is not limited to the examples given herein above. Any invention disclosed herein is embodied in each novel characteristic and each combination of characteristics, which particularly includes every combination of any features which are stated in the claims, even if this feature or this combination of features is not explicitly stated in the claims or in the examples.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term, “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

This patent application claims the priority of the European patent application 20315496.8, the disclosure content of which is hereby incorporated by reference.

### LIST OF REFERENCES

100 Autoinjector
110 Pouch
120 Narrowing portion
130 Gas sealing
140 Gas vessel
150 Delivery tube
160 Spout
170 Housing
175 Base
180 Button sealing
190 Needle carrier
200 Sealing part
210 Holding portion
220 Needle carrier sealing
230 Needle
240 Tube spring
250 Housing sealing
260 Button spring
270 Needle tube
280 Tube interface
290 Bridge
300 Button
310 Piercing spike
320 Window
330 Needle hole
y longitudinal axis

1. Autoinjector (100) comprising
a housing (170) which is gas-tight to the outside,
a fluid reservoir (110) with a fluid, wherein the fluid reservoir (110) comprises a flexible material,
a container (140) which comprises at least a first section which is configured to contain a first substance which comprises gas or which can be converted into gas, wherein
the container (140) is configured to assume two different states,
a first state in which the first section is sealed so that the at least first substance is contained in the first section,
a second state in which the at least first substance is released from the first section into the housing (170) causing an increase of gas pressure within the housing (170) which acts on the fluid reservoir (110) causing the fluid inside the fluid reservoir (110) to move through the fluid reservoir (110) towards and through an outlet (230) of the autoinjector (100).

2. Autoinjector according to claim 1, wherein the container (140) comprises a second section which is configured to contain a second substance which can be converted into gas, wherein the first section and the second section are separated by a separation member, such that when the separation member is opened the first substance, which comprises a substance which can be converted into gas, mixes with the second substance and generates gas.

3. Autoinjector according to claims 1 or 2, wherein the container (140) comprises a gas sealing (130) which seals the first and/or second section and which is opened when switching from the first state to the second state.

4. Autoinjector according to claim 3, comprising a trigger (300) which is movable along a longitudinal axis (y) from a first trigger position to a second trigger position, wherein
in the first trigger position the trigger (300) is separated from the gas sealing (130),
in the second trigger position the trigger (300) is in mechanical contact with the gas sealing (130), thereby opening the gas sealing (130), such that gas is releasable from the container (140) into the housing (170) and thereby increasing the gas pressure inside the housing (170), such that the fluid reservoir (110) is squeezed and the fluid inside the fluid reservoir (110) is moved to the outlet (230).

5. Autoinjector according to claim 4, comprising a trigger spring (260) which is mechanically connected to the trigger (300) such that when the trigger (300) moves from the first position to the second position the trigger moves the trigger spring (260) from a first position to a second position.

6. Autoinjector according to claims 4 or 5, wherein the trigger (300) is integrated into the housing (170).

7. Autoinjector according to any of the claims 4 to 6, wherein the trigger (300) comprises an element (310) which is configured to open the gas sealing (130).

8. Autoinjector according to any of the claims 4 to 7, comprising a trigger sealing (260) which seals the interface between the housing (170) and the trigger (300), to contribute to the housing (170) being gas-tight.

9. Autoinjector according to claim 2, wherein the separation member is configured to be opened during assembly of the autoinjector or by a tool.

10. Autoinjector according to any one of the preceding claims, comprising a delivery tube (150) which is configured to be in fluid communication with the fluid reservoir (110) and which is configured to be in fluid communication with the outlet (230), such that fluid from the fluid reservoir (110) is movable from the fluid reservoir through the delivery tube (150) to the outlet (230).

11. Autoinjector according to claims 4 and 10, comprising an outlet interface (270) which is movable relative the longitudinal axis (y) from a first interface position to a second interface position and which is in fluid communication with the outlet (230), wherein
in the first interface position the outlet interface (270) is not in fluid communication with the delivery tube (150),
in the second interface position the outlet interface (270) is in fluid communication with the delivery tube (150).

12. Autoinjector according to any of the preceding claims, wherein the outlet comprises a needle (230), which is movable along the longitudinal axis (y) from a first needle position to a second needle position, wherein
in the first needle position the needle (230) is completely contained in the housing (170),
in the second needle position at least a portion of the needle (230) has moved through a housing sealing (250).

13. Autoinjector (100) according to any of the preceding claims, wherein the housing (170) has a shape with a base which has a diameter larger than the height, which extends along the longitudinal axis (y).

14. Autoinjector (100) according to any of the preceding claims, wherein the fluid reservoir (110) comprises a medicament or drug.

15. Autoinjector according to any of the preceding claims, being a disposable or single-use device for providing a single dose.

16. Autoinjector according to any one of the preceding claims, wherein
the fluid reservoir (110) is arranged at a base (175) of the housing (170) along a circle segment,
the container (140) is arranged inside the housing (170) and at least partially circumferentially surrounded by the fluid reservoir (110).
